(19) United States
(12) Patent Application Publication
(10) Pub. No.: US 2026/0013733 A1
(43) Pub. Date: Jan. 15, 2026

(54) SYSTEMS AND METHODS FOR CONTINUOUS CARDIAC OUTPUT MONITORING

(71) Applicant: Becton, Dickinson and Company, Franklin Lakes, NJ (US)

(72) Inventors: Salvatore Manzella, JR., Aliso Viejo, CA (US); Oma Skyrus, Fountain Valley, CA (US)

(21) Appl. No.: 19/337,892

(22) Filed: Sep. 23, 2025

Related U.S. Application Data
(63) Continuation of application No. PCT/US2024/021209, filed on Mar. 22, 2024.
(60) Provisional application No. 63/492,179, filed on Mar. 24, 2023.

(51) Int. Cl. A61B 5/02; A61B 5/00; A61B 5/01; A61B 5/021; A61B 5/0215; A61B 5/1459; A61M 25/00; A61M 25/10

(52) U.S. Cl. CPC A61B 5/02028; A61B 5/01; A61B 5/02141; A61B 5/0215; A61B 5/1459; A61B 5/6853; A61B 5/7271; A61M 25/10; A61B 2560/0462; A61B 2562/0247; A61B 2562/0271; A61M 2025/0001; A61M 2025/1043; A61M 2205/3327; A61M 2205/3344; A61M 2205/3368; A61M 2210/125; A61M 2230/20; A61M 2230/30; A61M 2230/50

(57) ABSTRACT

Devices, systems, and methods provide measurements of continuous cardiac output (CCO). A pulmonary artery (PA) catheter—for example, a Swan-Ganz catheter—can be utilized to obtain multiple pressure measurements simultaneously from different locations within the circulatory system, such as in a pulmonary artery, right atrium, right ventricle, vena cava, etc. Continuous pressure measurements from multiple points can provide estimates of heart rate and stroke volume allowing computation of cardiac output along with each beat of the heart without the need for thermodilution.

### CROSS REFERENCE TO RELATED APPLICATIONS

This application is a continuation of International Application No. PCT/US2024/021209, filed Mar. 22, 2024, entitled “SYSTEMS AND METHODS FOR CONTINUOUS CARDIAC OUTPUT MONITORING,” which claims priority to U.S. Provisional Patent Application No. 63/492,179 entitled “Systems and Methods for Continuous Cardiac Output Monitoring,” filed Mar. 24, 2023, the disclosures of which are herein incorporated by reference.

### TECHNOLOGICAL FIELD

The disclosure is generally directed to pulmonic catheters, and more specifically catheters comprising multiple ports and lumens, which can determine cardiac output without thermodilution.

### BACKGROUND

Hemodynamic monitoring of patient routinely includes monitoring of blood pressure and many other informative hemodynamic parameters, such as cardiac output (CO), stroke volume (SV), and stroke volume variation (SVV). Each of these hemodynamic parameters are derivable from blood pressure waveforms and typically displayed by clinical monitors or otherwise made available to clinicians. Accurate calculation of hemodynamic parameters is of great importance to better inform clinicians about a patient's health and thus enable better informed decisions in treatment.

Cardiac output is an amount of blood pumped by the heart, and is closely matched to an individual's metabolic needs. Changes in CO from baseline are directly proportionate to changes in total body oxygen needs. During times of physiologic stress, cardiac output will increase to ensure adequate oxygen tissue perfusion. Mathematically, CO is the product of heart rate (HR) and stroke volume (SV) and is typically measured in liters per minute. HR is most commonly defined as the number of times the heart beats in one minute. SV is the volume of blood ejected during ventricular contraction or for each stroke of the heart.

Cardiac output is dependent on the health and capabilities of heart as well as the circulatory system-veins and arteries. Arterial compliance, vasoconstriction, and arterial pressure (afterload) directly affect stroke volume, and thus also affect CO. Since the circulatory system is a closed-loop system, CO is further dependent on the volume of blood reentering the heart from the veins, referred to as venous return (VR). VR also depends on the central venous pressure, which in turn is altered by venoconstriction.

Traditional methods of measuring CO include Fick's Principle to calculate CO based on oxygen exchange through a capillary bed, dynamic changes in mean arterial pressure and total peripheral resistance, pulse contour analysis, and thermodilution. Such methods can require specialized attachments or functions to a catheter and/or cannot provide a continuous measurement of cardiac output. Thus, there is a need for devices, systems, and methods that are capable of providing continuous cardiac output (CCO) measurements in a minimally invasive manner.

### SUMMARY

Systems and methods for hemodynamic monitoring can comprise utilization of a right-side pressure sensor to generate an arterial waveform. Hemodynamic data features can be extracted from the waveform. The extracted hemodynamic data features can be utilized in a computational model to identify specific parameters that can guide treatment.

In some aspects, the techniques described herein relate to a catheter for determining cardiac output, including a multi-lumen catheter body with an inflatable balloon defining a distal end and a plurality of connectors defining a proximal end, a first connector in the plurality of connectors connected to the balloon via a first lumen in the catheter body, a port for measuring pressure located on the catheter body located proximally from the distal end at a position such that the port is located in a right ventricle, when the balloon is located in a pulmonary artery, and a second connector in the plurality of connectors connected to the port via a second lumen in the catheter body.

In some aspects, the techniques described herein relate to a catheter, where the port is located at a position in the right ventricle where it is not affected by valvular motion.

In some aspects, the techniques described herein relate to a catheter, where the port is located approximately 7.5 cm to approximately 15 cm from the distal end.

In some aspects, the techniques described herein relate to a catheter, where the port is located approximately 12.7 cm from the distal end.

In some aspects, the techniques described herein relate to a catheter, further including an injectate port to provide an injectate location on the catheter body, and a third connector in the plurality of connectors connected to the injectate port via a third lumen in the catheter body.

In some aspects, the techniques described herein relate to a catheter, where the injectate port is selected from the group consisting of a distal lumen located at the distal end, a proximal injectate port located approximately 26 cm from the distal end, and a volume infusion port located approximately 30 cm from the distal end.

In some aspects, the techniques described herein relate to a catheter, further including a first injectate port to provide an injectate location on the catheter body, a third connector in the plurality of connectors connected to the first injectate port via a third lumen in the catheter body, a second injectate port to provide an injectate located on the catheter body, and a fourth connector in the plurality of connectors connected to the second injectate port via a fourth lumen in the catheter body.

In some aspects, the techniques described herein relate to a catheter, where the first and second injectate ports are selected from the group consisting of a distal lumen located at the distal end, a proximal injectate port located approximately 26 cm from the distal end, and a volume infusion port located approximately 30 cm from the distal end.

In some aspects, the techniques described herein relate to a catheter, further including a first injectate port to provide an injectate located on the catheter body, a third connector in the plurality of connectors connected to the first injectate port via a third lumen in the catheter body, a second injectate port to provide an injectate located on the catheter body, a fourth connector in the plurality of connectors connected to the second injectate port via a fourth lumen in the catheter body, a third injectate port to provide an injectate located on the catheter body, and a fifth connector in the plurality of connectors connected to the third injectate port via a fifth lumen in the catheter body.

In some aspects, the techniques described herein relate to a catheter where the first injectate port is a distal lumen located at the distal end, wherein the second injectate port is a proximal injectate port located approximately 26 cm from the distal end, and wherein the third injectate port is a volume infusion port located approximately 30 cm from the distal end.

In some aspects, the techniques described herein relate to a catheter, further including a thermal sensor to measure temperature located on the catheter body, and a sixth connector in the plurality of connectors connected to the thermal sensor via a sixth lumen in the catheter body.

In some aspects, the techniques described herein relate to a catheter, where the thermal sensor is selected from a thermistor and a thermocouple.

In some aspects, the techniques described herein relate to a catheter, further including a sensor for monitoring blood oxygen.

In some aspects, the techniques described herein relate to a catheter, further including a thermal filament located on the catheter body, and a seventh connector in the plurality of connectors connected to the thermal filament via a seventh lumen in the catheter body.

In some aspects, the techniques described herein relate to a method for continuous cardiac output determination including introducing a catheter to a pulmonary artery of an individual, obtaining a pressure reading from the catheter, and determining a cardiac output (CO) of the individual based on the pressure reading from the catheter.

In some aspects, the techniques described herein relate to a method, where introducing the catheter includes advancing the catheter through a right atrium, a tricuspid valve, a right ventricle, a pulmonary valve, and a pulmonary artery of the individual.

In some aspects, the techniques described herein relate to a method, where introducing the catheter includes accessing a femoral vein of the individual and advancing the catheter via an inferior vena cava to a right atrium of an individual.

In some aspects, the techniques described herein relate to a method, further including providing medical intervention to the individual based on the CO of the individual.

In some aspects, the techniques described herein relate to a method, where providing medical intervention includes administering a compound to increase CO.

In some aspects, the techniques described herein relate to a method, where the compound increases heart rate or stroke volume.

In some aspects, the techniques described herein relate to a method, where providing medical intervention includes administering a compound to decrease CO.

In some aspects, the techniques described herein relate to a method, where the compound decreases heart rate or stroke volume.

In some aspects, the techniques described herein relate to a method, further including removing the catheter from the individual.

In some aspects, the techniques described herein relate to a method, where the catheter includes a multi-lumen catheter body with an inflatable balloon defining a distal end and a plurality of connectors defining a proximal end, a first connector in the plurality of connectors connected to the balloon via a first lumen in the catheter body, a port for measuring pressure located on the catheter body located proximally from the distal end at a position such that the port is located in a right ventricle, when the balloon is located in a pulmonary artery, and a second connector in the plurality of connectors connected to the port via a second lumen in the catheter body.

In some aspects, the techniques described herein relate to a method, where the port is located at a position in the right ventricle where it is not affected by valvular motion.

In some aspects, the techniques described herein relate to a method, where the port is located approximately 7.5 cm to approximately 15 cm from the distal end.

In some aspects, the techniques described herein relate to a method, where the port is located approximately 12.7 cm from the distal end.

In some aspects, the techniques described herein relate to a method, further including an injectate port to provide an injectate located on the catheter body, and a third connector in the plurality of connectors connected to the injectate port via a third lumen in the catheter body.

In some aspects, the techniques described herein relate to a method, where the injectate port is selected from the group consisting of a distal lumen located at the distal end, a proximal injectate port located approximately 26 cm from the distal end, and a volume infusion port located approximately 30 cm from the distal end.

In some aspects, the techniques described herein relate to a method, further including a first injectate port to provide an injectate located on the catheter body, a third connector in the plurality of connectors connected to the first injectate port via a third lumen in the catheter body, a second injectate port to provide an injectate located on the catheter body, and a fourth connector in the plurality of connectors connected to the second injectate port via a fourth lumen in the catheter body.

In some aspects, the techniques described herein relate to a method, where the first and second injectate ports are selected from the group consisting of a distal lumen located at the distal end, a proximal injectate port located approximately 26 cm from the distal end, and a volume infusion port located approximately 30 cm from the distal end.

In some aspects, the techniques described herein relate to a method, further including a first injectate port to provide an injectate located on the catheter body, a third connector in the plurality of connectors connected to the first injectate port via a third lumen in the catheter body, a second injectate port to provide an injectate located on the catheter body, a fourth connector in the plurality of connectors connected to the second injectate port via a fourth lumen in the catheter body, a third injectate port to provide an injectate located on the catheter body, and a fifth connector in the plurality of connectors connected to the third injectate port via a fifth lumen in the catheter body.

In some aspects, the techniques described herein relate to a method where the first injectate port is a distal lumen located at the distal end, wherein the second injectate port is a proximal injectate port located approximately 26 cm from the distal end, and wherein the third injectate port is a volume infusion port located approximately 30 cm from the distal end.

In some aspects, the techniques described herein relate to a method, further including a thermal sensor to measure temperature located on the catheter body, and a sixth connector in the plurality of connectors connected to the thermal sensor via a sixth lumen in the catheter body.

In some aspects, the techniques described herein relate to a method, where the thermal sensor is selected from a thermistor and a thermocouple.

In some aspects, the techniques described herein relate to a method, further including a sensor for monitoring blood oxygen.

In some aspects, the techniques described herein relate to a method, further including a thermal filament located on the catheter body, and a seventh connector in the plurality of connectors connected to the thermal filament via a seventh lumen in the catheter body.

### BRIEF DESCRIPTION OF THE DRAWINGS

The description and claims will be more fully understood with reference to the following figures, which are presented as examples of the disclosure and should not be construed as a complete recitation of the scope of the disclosure.

FIG. 1A illustrates an example of a pulmonary catheter that includes a balloon defining a distal end and a proximal end defined by one or more connectors.

FIG. 1B illustrates a perspective view of a cross-section of a catheter body.

FIG. 2A provides an exemplary catheter that includes a port located at a position within the right ventricle.

FIG. 2B illustrates a cross-sectional view of a catheter body.

FIG. 3 illustrates a flow chart of a method of determining cardiac output.

FIG. 4 illustrates a block diagram of components of a processing system in a computing device that can be used to determine cardiac output.

FIG. 5 illustrates a network diagram of a distributed system to determine cardiac output.

### DETAILED DESCRIPTION

The current disclosure details devices, systems, and methods to provide measurements of continuous cardiac output (CCO). A set of sensors capable of obtaining multiple pressure measurements simultaneously can be utilized for capturing hemodynamic parameters for determining CCO. In one specific implementation, a set of sensors is provided via a pulmonary artery (PA) catheter—for example, a Swan-Ganz catheter—to capture hemodynamic parameters for determining CCO. The multiple pressure measurements can be obtained from a variety of different locations within the circulatory system, including a pulmonary artery, right atrium, right ventricle, vena cava, etc. Continuous capture of pressure measurements from multiple locations within the circulatory system can provide estimates of heart rate and stroke volume, allowing computation of cardiac output along with each beat of the heart.

The described systems, devices, and methods should not be construed as limiting in any way. Instead, the present disclosure is directed toward all novel and nonobvious features and aspects of the various disclosed systems and devices, alone and in various combinations and sub-combinations with one another. The disclosed systems, devices, and methods are not limited to any specific aspect, feature, or combination thereof, nor do the disclosed systems, devices, and methods require that any one or more specific advantages be present or problems be solved.

Various examples of CCO monitoring systems and components thereof are disclosed herein, and any combination of these examples can be made unless specifically excluded. The different constructions and features of CCO monitoring systems can be mixed and matched, such as by combining any tool for accessing the heart and/or monitoring CO and CCO, even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

The terms “proximal” and “distal” as used throughout the description relate to a catheter system axis, where the end where the procedure is performed is the distal end and the opposite end where the catheter system is controlled is the proximal end. Accordingly, the distal end of the catheter system is the leading end that first traverses into the body and first reaches the procedure site. Conversely, the proximal end of the catheter system is the portion that remains extracorporeal. Likewise, a distal movement along the catheter axis would be movement of a component in a direction towards (or beyond) a site of procedure and a proximal movement along the catheter axis would be movement of a component in an opposite direction. Although these terms have a relationship with a site of procedure, it is to be understood that these terms are used for reference and the site of procedure does not need to be present when interpreting the components or movements of the devices and systems described herein.

Many catheters used for measuring hemodynamic parameters, including PA catheters (e.g., Swan-Ganz catheters), possess multiple lumens, sensors, ports, etc., allowing for measurement in multiple locations. These multiple components are utilized to measure localized pressure, heart beats, thermal dilution, blood oxygen, etc. Examples of such catheters are described in U.S. Pat. No. 3,995,623; US 2017/0027458; US 2018/0000421; WO 2021/062527; the disclosures of which are hereby incorporated by reference in their entireties.

Catheters of the current disclosure allow for pressure readings within multiple locations simultaneously, which can be combined to provide a continuous measurement of an individual's cardiac output. One such solution is to obtain pressure readings from within the pulmonary artery and right ventricle simultaneously to determine CCO, allowing for determination of CCO without thermodilution. Accordingly, in some implementations, a set of sensor for determining CCO does not require thermal ports and thermistors (or other thermal sensor such as a thermocouple) to calculate CCO via thermodilution. In some implementations, a set of sensor for determining CCO does not include thermal sensors, which can reduce size and/or complexity of a catheter. Additionally, because thermodilution requires introduction of an injectate to obtain a measurement, obviating thermodilution as a methodology reduces potential complications caused by introduction of said injectate, such as contamination or infection. Accordingly, in some implementations, a method to determine CCO does not require introduction of an injectate. And in some implementations, a method to determine CCO does not include introduction of an injectate.

Notably, placement of a pressure-reading port within a chamber of the heart can be problematic due to effects or perturbations caused by valvular action (e.g., opening and closing of valves as related to systole and diastole). Thus, the placement of such a port should lie at a point that has minimal interference from local physiology, such as valves, walls, etc. that can cause aberrations or errors in CCO measurements. As described herein, an additional port can be placed on a catheter body at a position as to be unaffected by valvular action and/or valvular motion.

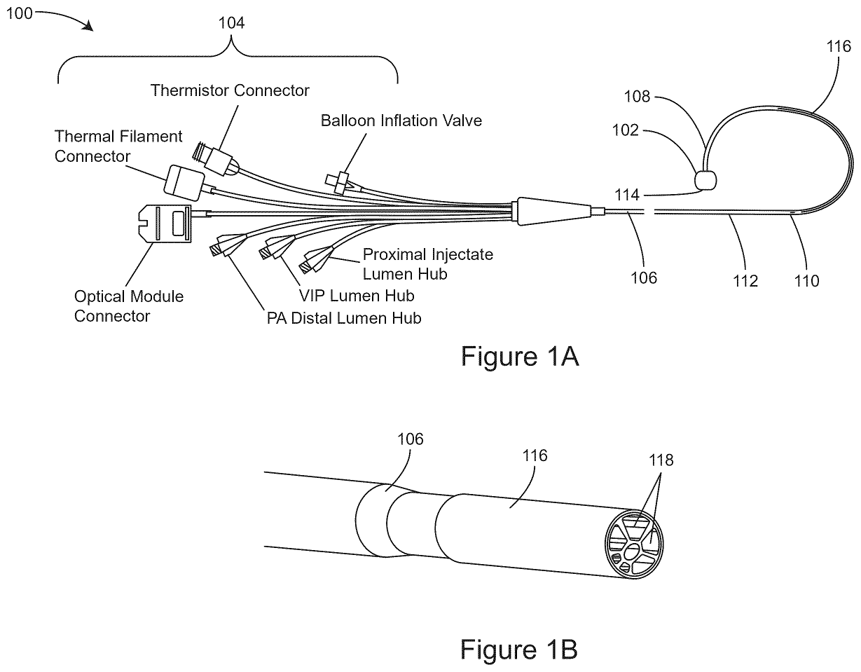

FIG. 1A provides an example of a prior art pulmonary catheter 100 that includes a balloon 102 defining a distal end and a proximal end defined by one or more connectors 104. The balloon 102 allows the catheter 100 to be floated into position in a pulmonary artery of an individual to perform various procedures, such as acquiring a pulmonary capillary wedge pressure (PCSWP).

A multi-lumen body 106 defines the catheter between the connectors 104 and balloon 102. As described previously, body 106 can include various ports, sensors, etc. Catheter 100 possesses a thermistor 108 for measuring temperature located approximately 4 cm proximal from the distal end and an injectate port 110 located at approximately 26 cm from distal end (i.e., toward proximal end). In various implementations, catheter 100 can further include additional ports (e.g., volume infusion port (VIP) 112, distal lumen port 114) to allow infusion at different positions. As illustrated, the distal lumen port 114 is located at the distal end, allowing for infusion downstream of the catheter, while VIP port 112 is located more proximally (illustrated at approximately 30 cm from distal end) than injectate port 110 for infusion into another location (e.g., atrium, vena cava, etc.). Catheter 100 further includes a thermal filament 116 that can be used to adjust temperature in the environment around the catheter, including during thermodilution. Such temperature adjustment can include induction, Pelletier effect, and/or any other method of adjusting thermal energy in a system.

The inclusion of a thermal filament 116 restricts the ability for catheter 100 to include a port along the length filament. Because thermal filaments can be quite long, when performing a procedure such as PCWP, it can extend through most or all of a right ventricle (e.g., when balloon 102 is in the pulmonary artery). Additionally, thermal filaments can restrict flexibility of a catheter and/or reduce diameter of the catheter body. FIG. 1B illustrates a perspective view of a cross-section of a body 106. As illustrated, in order to maintain a constant diameter on body 106, the diameter of the lumens 118 must be reduced in the positions of thermal filament 116. While reducing diameter generally does not affect flow rate, the reduction can cause increased fluid velocity, greater chance of obstruction, and/or other problems associated with smaller cross-sectional areas.

Each of the ports, sensors, components in catheter 100 is connected to a connector 104, which can be connected to a monitor, controller, valve, fluid reservoir, and/or other proximal device. Monitors can provide visual, audial, and/or other feedback to a medical provider during a procedure, such as directly measured parameters (e.g., temperature, heart rate, blood oxygen, pressures, etc.) or derived parameters (e.g., cardiac output). A controller can provide specific instructions to devices to control a thermal filament 116, inflation of balloon 102, and/or infusion via one or more of injectate port 110, VIP port 112, and/or distal lumen port 114). Valves and/or reservoirs can be used to further control infusion, inflation, etc.

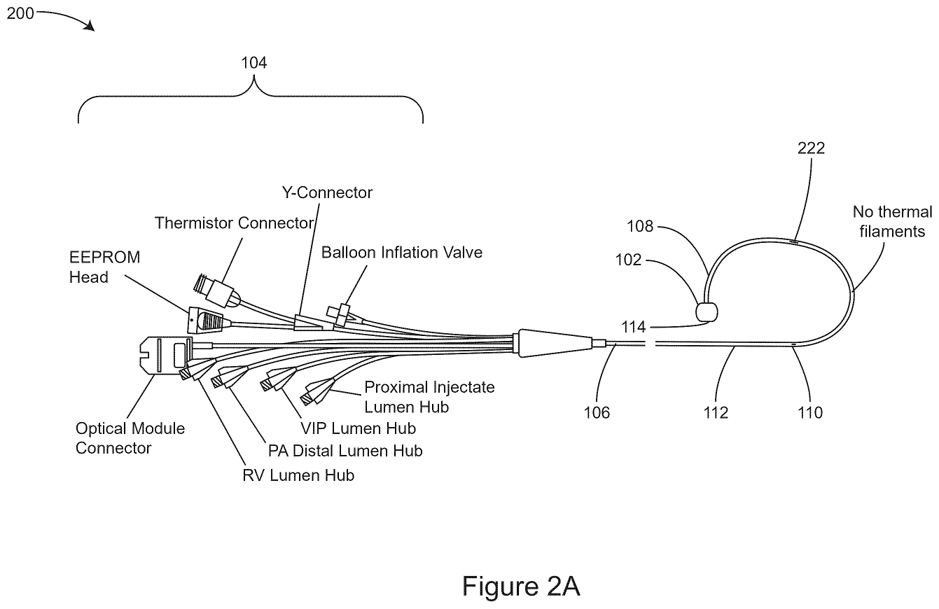

To resolve many of the problems above, various catheters of the disclosure omit a thermal filament 116 and instead is replace by one or more additional ports to measure pressure. FIG. 2A provides an example of a catheter 200 that includes a port 222 located at a position within the right ventricle. Port 222 allows for pressure measurements in both a right ventricle and pulmonary artery that can be used for CCO monitoring in a safer and/or more cost-effective manner. Unless noted otherwise, features illustrated in FIG. 2A are the same or share the same or functionally similar purpose as the similarly numbered features in FIG. 1A. In catheter 200, omission of thermal filament allows one lumen in body 106 to be used for port 222. Additionally, a connector for a thermal filament can be replaced with a connector for port 222. Certain catheters 200 include an EEPROM head as a connector 104. Such EEPROM does not require connection to a lumen of body 106, but can be attached to another connector 104 for convenience to a medical professional. Catheter 200 can be connected to a monitor, a controller, a valve, and/or a fluid reservoir as described above in regard to catheter 100.

As previously mentioned, to obtain an accurate measurement of right ventricular pressure from port 222, the positioning of port 222 should be at a distance far enough from pulmonic and/or tricuspid valves within the heart as to avoid any interference and/or perturbations caused by valvular movement or action. Such a distance can be determined empirically via by measuring distances between valves and/or by the influence of valvular action on pressure readings from a port. In general, the distance of port 222 from distal end of the catheter can be approximately 7.5 cm (e.g., +2.5 cm) to approximately 15 cm (e.g., +5 cm). In some specific implementations, port 222 is positioned approximately 12.7 cm (e.g., +0.1 cm) from the distal end (e.g., toward proximal end). At a distance of approximately 12.7 cm, port 222 is greater than 6 standard deviations away from both a pulmonic and tricuspid valve, such that the influence of valvular actions is minimized.

As different components or features of catheter 200 may have different spatial requirements based on throughput (e.g., different viscosities of fluids, wire size for a particular resistance or transmission, etc.) As such, FIG. 2B provides an exemplary cross-sectional view of the lumens within body 106 of catheter 200. As provided in FIG. 2B, cross-sectional lumen areas vary depending on purpose. For example, lumens for providing an injectate, infusion, and/or pressure measurement are generally larger, including a right ventricle (RV) lumen 250 connected to an RV port 222; a proximal injectate lumen 252, which is connected to proximal injectate port 110; proximal infusion lumen 254, which is connected to VIP port 112; and pulmonary artery (PA) distal lumen 256 connected to a distal lumen port 114. In contrast, lumens for gases or electronics tend to be smaller, such as inflation lumen 258 connected to balloon 102 and thermistor wire lumen 260 connected to a thermistor 108. One or more lumens can be used for additional components, including sensors. For example, central lumen 262 can be used to contain fibers or fiber optics to transmit light energy. In some implementations, fiber optics can be used in conjunction with an optical module to provide light and/or detect spectroscopic measurements from a catheter 200 (e.g., for blood oxygen measurements).

### Methods for CCO Determination

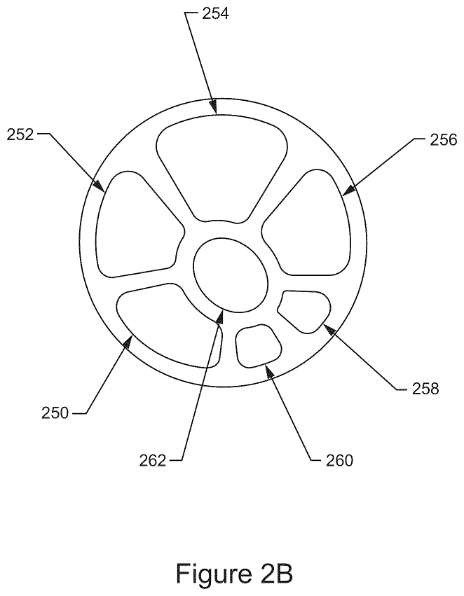

Various implementations apply procedures herein into a practical application of monitoring CCO of an individual. Many such applications follow a process similar to process 300 illustrated in FIG. 3. Process 300 is an exemplary process that can be used to determine CCO for an individual and optionally provide a medical intervention (e.g., treatment) for the individual based on the CO.

At 302, many processes introduce a catheter to the pulmonary artery of an individual. Such catheterization can include minimally invasive methods, such as percutaneous introduction. Generally, any vascular path that leads to the pulmonary artery may be used. Percutaneous introduction can include accessing a vein, inserting the catheter (e.g., catheter 100 or catheter 200), and advancing the catheter to the pulmonary artery. This path can include the femoral vein, inferior vena cava, and right atrium, tricuspid valve, right ventricle, and pulmonary valve into the pulmonary artery. In certain instances, a balloon on the catheter can be inflated to assist in floating the catheter into position. Visualization and/or imaging tools, including X-ray, MRI, CT, etc. can be used to monitor the position of a catheter as it is advanced through an individual.

Once a catheter is in position, pressure readings can be obtained at 304. Such pressure readings can be obtained from one or more pressure sensor on the catheter, including a pressure sensor attached to a port (e.g., VIP port 112, distal lumen port 114, port 222, injectate port 110) or a pressure sensor attached to balloon 102. The pressure readings may be processed on a clinical monitor.

At 308, using the pressure readings, CO can be determined. In many instances, a clinical monitor can utilize an algorithm, such as a machine learning algorithm to determine CO of the individual. Such CO determination can be displayed visually on a display in communication with the monitor. Such displays can be local (i.e., physically attached other monitor) or remote (i.e., data is transmitted via a network or other communication system to the display). Due to the pressure readings being unaffected by valvular actions (e.g., in catheter 200), continual pressure measurements can be obtained to update CO continuously, including pressure readings obtained at a frequency of 1 Hz or less (e.g., 1 Hz, 2 Hz, 3 Hz, 4 Hz, 5 Hz, 10 Hz, 15 Hz, 20 Hz, 25 Hz, 50 Hz, 100 Hz, or more).

Depending on CO, medical intervention can be provided to the individual at 306. For example, if CO falls below a threshold, administering one or more medical compounds to boost heart rate or stroke volume can be used to increase CO. Similarly, if CO rises above a certain threshold, administering one or more compounds can be utilized to reduce heart rate or stroke volume.

At 310, the catheter can be removed from the individual. In many instances, catheter removal can follow a known process, such as by deflating balloon 102, then retracting catheter through the individual's body until it is completely removed. Once removed, any access point (e.g., femoral vein) can be sealed to prevent blood loss, infection, etc. Sealing can include suturing, medical adhesives, and/or any other method, technique, or system for closing an access point.

### Computer Executed Implementations

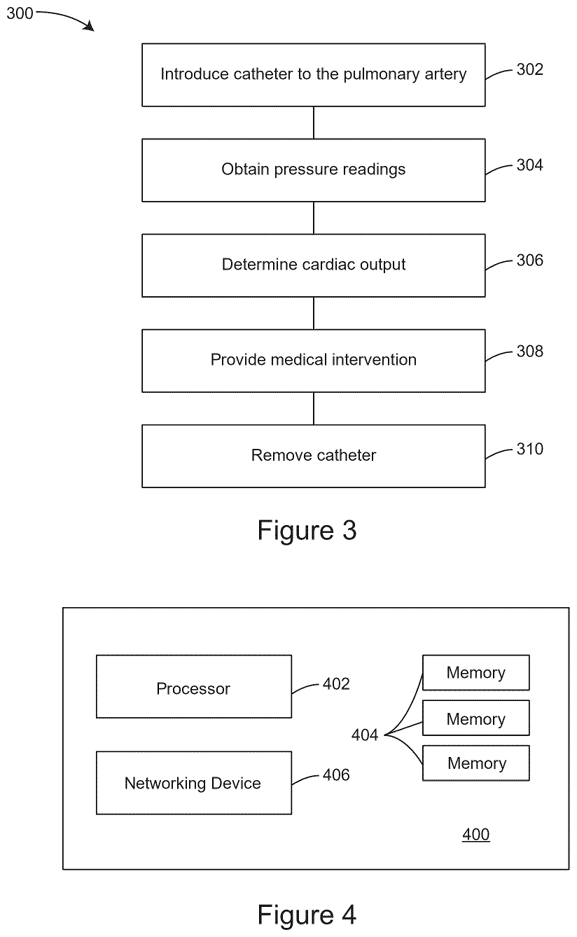

Processes that provide the methods and systems for determining cardiovascular parameters in accordance with some implementations are executed by a computing device or computing system, such as a desktop computer, tablet, mobile device, laptop computer, notebook computer, server system, and/or any other device capable of performing one or more features, functions, methods, and/or steps as described herein. The relevant components in a computing device that can perform the processes are shown in FIG. 4. One skilled in the art will recognize that computing devices or systems may include other components that are omitted for brevity without departing from described implementations. A computing device 400 can comprises a processor 402 and at least one memory 404. Memory 404 can be a non-volatile memory and/or a volatile memory, and the processor 402 is a processor, microprocessor, controller, or a combination of processors, microprocessor, and/or controllers that performs instructions stored in memory 404. Such instructions stored in the memory 404, when executed by the processor, can direct the processor, to perform one or more features, functions, methods, and/or steps as described herein. Any input information or data can be stored in the memory 404—either the same memory or another memory. The computing device 400 may have hardware and/or firmware that can include the instructions and/or perform these processes.

Certain computing devices can include a networking device 406 to allow communication (wired, wireless, etc.) to another device, such as through a network, near-field communication, Bluetooth, infrared, radio frequency, and/or any other suitable communication system. Such systems can be beneficial for receiving data, information, or input (e.g., images) from another computing device and/or for transmitting data, information, or output (e.g., quality score, rating, etc.) to another device. The networking device can be used to send and/or receive update models, interfaces, etc. to a user device.

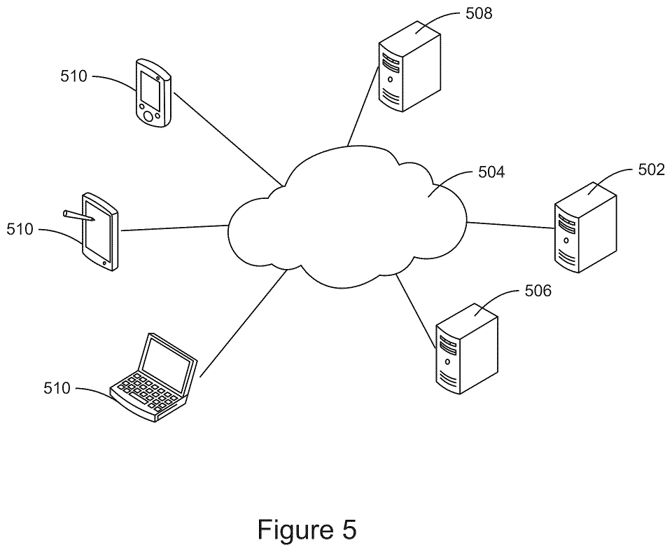

Turning to FIG. 5, a distributed computing device that can be utilized is illustrated. Distributed computing devices may be useful where computing power is not possible at a local level, and a central computing device (e.g., server) performs one or more features, functions, methods, and/or steps described herein. A computing device 502 (e.g., server) can be connected to a network 504 (wired and/or wireless), where it can receive inputs from one or more computing devices, including data from a records database or repository 506, data provided from a laboratory computing device 508, and/or any other relevant information from one or more other remote devices 510. Once computing device 502 performs one or more features, functions, methods, and/or steps described herein, any outputs can be transmitted to one or more computing devices 506, 508, 510 for entering into records.

In additional implementations, the instructions for the processes can be stored in any of a variety of non-transitory computer readable media appropriate to a specific application.

### Examples

Example 1. A catheter for determining cardiac output, comprising:
a multi-lumen catheter body with an inflatable balloon defining a distal end and a plurality of connectors defining a proximal end;
a first connector in the plurality of connectors connected to the balloon via a first lumen in the catheter body;
a port for measuring pressure located on the catheter body located proximally from the distal end at a position such that the port is located in a right ventricle, when the balloon is located in a pulmonary artery; and
a second connector in the plurality of connectors connected to the port via a second lumen in the catheter body.

Example 2. The catheter of example 1, wherein the port is located at a position in the right ventricle where it is not affected by valvular motion.

Example 3. The catheter of example 1 or 2, wherein the port is located approximately 7.5 cm to approximately 15 cm from the distal end.

Example 4. The catheter of example 3, wherein the port is located approximately 12.7 cm from the distal end.

Example 5. The catheter of any of examples 1-4, further comprising:
an injectate port to provide an injectate location on the catheter body; and
a third connector in the plurality of connectors connected to the injectate port via a third lumen in the catheter body.

Example 6. The catheter of example 5, wherein the injectate port is selected from the group consisting of: a distal lumen located at the distal end, a proximal injectate port located approximately 26 cm from the distal end, and a volume infusion port located approximately 30 cm from the distal end.

Example 7. The catheter of any of examples 1-4, further comprising:
a first injectate port to provide an injectate location on the catheter body;
a third connector in the plurality of connectors connected to the first injectate port via a third lumen in the catheter body;
a second injectate port to provide an injectate located on the catheter body; and
a fourth connector in the plurality of connectors connected to the second injectate port via a fourth lumen in the catheter body.

Example 8. The catheter of example 7, wherein the first and second injectate ports are selected from the group consisting of: a distal lumen located at the distal end, a proximal injectate port located approximately 26 cm from the distal end, and a volume infusion port located approximately 30 cm from the distal end.

Example 9. The catheter of any of examples 1-4, further comprising:
a first injectate port to provide an injectate located on the catheter body;
a third connector in the plurality of connectors connected to the first injectate port via a third lumen in the catheter body;
a second injectate port to provide an injectate located on the catheter body;
a fourth connector in the plurality of connectors connected to the second injectate port via a fourth lumen in the catheter body;
a third injectate port to provide an injectate located on the catheter body; and
a fifth connector in the plurality of connectors connected to the third injectate port via a fifth lumen in the catheter body.

Example 10. The catheter of example 9:
wherein the first injectate port is a distal lumen located at the distal end;
wherein the second injectate port is a proximal injectate port located approximately 26 cm from the distal end; and
wherein the third injectate port is a volume infusion port located approximately 30 cm from the distal end.

Example 11. The catheter of any of examples 1-10, further comprising a thermal sensor to measure temperature located on the catheter body, and a sixth connector in the plurality of connectors connected to the thermal sensor via a sixth lumen in the catheter body.

Example 12. The catheter of example 11, wherein the thermal sensor is selected from a thermistor and a thermocouple.

Example 13. The catheter of any of examples 1-12, further comprising a sensor for monitoring blood oxygen.

Example 14. The catheter of any of examples 1-10, wherein the multi-lumen catheter body lacks a thermal sensor.

Example 15. A method for continuous cardiac output determination comprising:
introducing a catheter to a pulmonary artery of an individual;
obtaining a pressure reading from the catheter; and determining a cardiac output (CO) of the individual based on the pressure reading from the catheter.

Example 16. The method of example 15, wherein introducing the catheter comprises advancing the catheter through a right atrium, a tricuspid valve, a right ventricle, a pulmonary valve, and a pulmonary artery of the individual.

Example 17. The method of example 15 or 16, wherein introducing the catheter comprises accessing a femoral vein of the individual and advancing the catheter via an inferior vena cava to a right atrium of an individual.

Example 18. The method of any of examples 15-17, further comprising providing medical intervention to the individual based on the CO of the individual.

Example 19. The method of example 18, wherein providing medical intervention comprises administering a compound to increase CO.

Example 20. The method of example 19, wherein the compound increases heart rate or stroke volume.

Example 21. The method of example 18, wherein providing medical intervention comprises administering a compound to decrease CO.

Example 22. The method of example 21, wherein the compound decreases heart rate or stroke volume.

Example 23. The method of any of examples 15-22, further comprising removing the catheter from the individual.

Example 24. The method of any of examples 15-23, wherein the catheter comprises: a multi-lumen catheter body with an inflatable balloon defining a distal end and a plurality of connectors defining a proximal end;
a first connector in the plurality of connectors connected to the balloon via a first lumen in the catheter body;
a port for measuring pressure located on the catheter body located proximally from the distal end at a position such that the port is located in a right ventricle, when the balloon is located in a pulmonary artery; and
a second connector in the plurality of connectors connected to the port via a second lumen in the catheter body.

Example 25. The method of example 24, wherein the port is located at a position in the right ventricle where it is not affected by valvular motion.

Example 26. The method of example 24 or 25, wherein the port is located approximately 7.5 cm to approximately 15 cm from the distal end.

Example 27. The method of any of examples 24-26, wherein the port is located approximately 12.7 cm from the distal end.

Example 28. The method of any of examples 24-27, further comprising:
an injectate port to provide an injectate located on the catheter body; and a third connector in the plurality of connectors connected to the injectate port via a third lumen in the catheter body.

Example 29. The method of example 28, wherein the injectate port is selected from the group consisting of: a distal lumen located at the distal end, a proximal injectate port located approximately 26 cm from the distal end, and a volume infusion port located approximately 30 cm from the distal end.

Example 30. The method of any of examples 24-27, further comprising:
a first injectate port to provide an injectate located on the catheter body;
a third connector in the plurality of connectors connected to the first injectate port via a third lumen in the catheter body;
a second injectate port to provide an injectate located on the catheter body; and
a fourth connector in the plurality of connectors connected to the second injectate port via a fourth lumen in the catheter body.

Example 31. The method of example 30, wherein the first and second injectate ports are selected from the group consisting of: a distal lumen located at the distal end, a proximal injectate port located approximately 26 cm from the distal end, and a volume infusion port port located approximately 30 cm from the distal end.

Example 32. The method of any of examples 24-27, further comprising:
a first injectate port to provide an injectate located on the catheter body;
a third connector in the plurality of connectors connected to the first injectate port via a third lumen in the catheter body;
a second injectate port to provide an injectate located on the catheter body;
a fourth connector in the plurality of connectors connected to the second injectate port via a fourth lumen in the catheter body;
a third injectate port to provide an injectate located on the catheter body; and
a fifth connector in the plurality of connectors connected to the third injectate port via a fifth lumen in the catheter body.

Example 33. The method of example 32:
wherein the first injectate port is a distal lumen located at the distal end;
wherein the second injectate port is a proximal injectate port located approximately 26 cm from the distal end; and
wherein the third injectate port is a volume infusion port located approximately 30 cm from the distal end.

Example 34. The method of any of examples 24-33, further comprising a thermal sensor to measure temperature located on the catheter body, and a sixth connector in the plurality of connectors connected to the thermal sensor via a sixth lumen in the catheter body.

Example 35. The method of example 34, wherein the thermal sensor is selected from a thermistor and a thermocouple.

Example 36. The method of any of examples 24-35, further comprising a sensor for monitoring blood oxygen.

Example 37. The method of any of examples 24-33, wherein the multi-lumen catheter body lacks a thermal sensor.

### DOCTRINE OF EQUIVALENTS

Having described several implementations, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred implementations of the present invention are merely illustrative of the invention as a whole, and that variations in the components or steps of the present invention may be made within the spirit and scope of the invention. Accordingly, the present invention is not limited to the specific implementations described herein, but, rather, is defined by the scope of the appended claims.

What is claimed is:

1. A catheter for determining cardiac output, comprising:
a multi-lumen catheter body with an inflatable balloon defining a distal end and a plurality of connectors defining a proximal end;
a first connector of the plurality of connectors connected to the balloon via a first lumen in the catheter body;
a port for measuring pressure located on the catheter body proximally from the distal end at a position such that the port is configured to be located in a right ventricle when the balloon is located in a pulmonary artery; and
a second connector of the plurality of connectors connected to the port via a second lumen in the catheter body.

2. The catheter of claim 1, wherein the port is configured to be located at a position in the right ventricle where it is not affected by valvular motion.

3. The catheter of claim 1, wherein the port is located approximately 7.5 cm to approximately 15 cm from the distal end.

4. The catheter of claim 3, wherein the port is located approximately 12.7 cm from the distal end.

5. The catheter of claim 1, further comprising:
an injectate port to provide an injectate located on the catheter body; and
a third connector of the plurality of connectors connected to the injectate port via a third lumen in the catheter body.

6. The catheter of claim 5, wherein the injectate port is selected from the group consisting of: a distal lumen located at the distal end, a proximal injectate port located approximately 26 cm from the distal end, and a volume infusion port located approximately 30 cm from the distal end.

7. The catheter of claim 1, further comprising:
a first injectate port to provide an injectate located on the catheter body;
a third connector of the plurality of connectors connected to the first injectate port via a third lumen in the catheter body;
a second injectate port to provide an injectate located on the catheter body; and
a fourth connector of the plurality of connectors connected to the second injectate port via a fourth lumen in the catheter body.

8. The catheter of claim 7, wherein the first and second injectate ports are selected from the group consisting of: a distal lumen located at the distal end, a proximal injectate port located approximately 26 cm from the distal end, and a volume infusion port located approximately 30 cm from the distal end.

9. The catheter of claim 1, further comprising:
a first injectate port to provide an injectate located on the catheter body;
a third connector of the plurality of connectors connected to the first injectate port via a third lumen in the catheter body;
a second injectate port to provide an injectate located on the catheter body;
a fourth connector of the plurality of connectors connected to the second injectate port via a fourth lumen in the catheter body;
a third injectate port to provide an injectate located on the catheter body; and
a fifth connector of the plurality of connectors connected to the third injectate port via a fifth lumen in the catheter body.

10. The catheter of claim 9:
wherein the first injectate port is a distal lumen located at the distal end;
wherein the second injectate port is a proximal injectate port located approximately 26 cm from the distal end; and
wherein the third injectate port is a volume infusion port located approximately 30 cm from the distal end.

11. The catheter of claim 10, further comprising a thermal sensor to measure temperature located on the catheter body, and a sixth connector of the plurality of connectors connected to the thermal sensor via a sixth lumen in the catheter body.

12. The catheter of claim 11, wherein the thermal sensor is selected from a thermistor and a thermocouple.

13. The catheter of claim 1, further comprising a sensor for monitoring blood oxygen.

14. The catheter of claim 1, wherein the multi-lumen catheter body lacks a thermal sensor.

15. A method for continuous cardiac output determination comprising:
introducing a catheter to a pulmonary artery of an individual;
obtaining a pressure reading from the catheter; and
determining a cardiac output (CO) of the individual based on the pressure reading from the catheter.

16. The method of claim 15, wherein introducing the catheter comprises advancing the catheter through a right atrium, a tricuspid valve, a right ventricle, a pulmonary valve, and a pulmonary artery of the individual.

17. The method of claim 15, wherein the step of obtaining a pressure reading comprises utilizing a port for measuring pressure, wherein the catheter comprises:
a multi-lumen catheter body with an inflatable balloon defining a distal end and a plurality of connectors defining a proximal end;
a first connector of the plurality of connectors connected to the balloon via a first lumen in the catheter body;
the port for measuring pressure located on the catheter body proximally from the distal end at a position such that the port is located in a right ventricle when the balloon is located in a pulmonary artery; and
a second connector of the plurality of connectors connected to the port via a second lumen in the catheter body.

18. The method of claim 17, wherein the port is located at a position in the right ventricle where it is not affected by valvular motion.

19. The method of claim 17, wherein the port is located approximately 7.5 cm to approximately 15 cm from the distal end.

20. The method of claim 19, wherein the port is located approximately 12.7 cm from the distal end.
